(19) United States
(12) Patent Application Publication
(10) Pub. No.: US 2022/0023192 A1
(43) Pub. Date: Jan. 27, 2022

(54) Lip Plumper

(71) Applicant: Xu Xiaolan, Zhengzhou (CN)

(72) Inventor: Xu Xiaolan, Zhengzhou (CN)

(21) Appl. No.: 16/939,089

(22) Filed: Jul. 27, 2020

(51) Int. Cl. A61K 8/92; A61K 8/37; A61K 8/34; A61K 8/81; A61Q 19/00

(52) U.S. Cl. CPC A61K 8/92; A61K 8/37; A61K 8/8111; A61K 8/8117; A61Q 19/001; A61K 8/34

(57) ABSTRACT

An effective treatment method for improving the appearance of lip resulting in lips enhancement and enlargement. The method comprising of treating the lip area with the proposed Lip Plumper Lip Gloss resulting enhanced hydration for lips and simultaneously plumped effect with shine and diluted unattractive wrinkles.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

This invention relates to generally to devices and methods to enhance human lips. This invention relates particularly to a Lip Plumper Lip Gloss device which enhances the shape of lips and make them smoother and softer.

### Description of the Related Art

Lip size and shape are facial features, which directly impact attractiveness levels. Many believe a person's lips and smile to be an indicator of beauty. Full lips, especially in Western cultures, are considered attractive. Some suggest that the trend towards fuller lips developed because lips become less full with age. As one ages, gravitational effects and atrophy of tissue and muscles on and surrounding the lips often lead to lips which appear deflated and disproportionate. Fuller lips, however, are often associated with a healthy lifestyle, youth, and beauty.

In one study, it was found that men give more attention to women with fuller lips. Women having “luscious pouts” were found to attract more attention from men compared to thin-lipped women. In this same study, lips were found to represent one of the most sensual areas on a woman's body and serve as a biological indicator of a woman's health and fertility.

Because of the trend toward fuller lips, many go to considerable lengths to enhance lip shape and size. Therefore, several procedures and devices have been developed to enlarge lips and change lip shape. Lip augmentation, for example, is used frequently in surgical and non-surgical procedures to increase lip fullness. These procedures include injecting and implanting various types of materials into the lips.

But many risks are associated with lip augmentation. Lips can be overfilled with injected materials. Allergic reactions to anesthetics and injected and implanted materials can occur, resulting in redness, swelling, itching, etc. Abnormal lip shapes may develop, e.g. trout pout, duck lips, sausage lips, fish lips, etc. Bruising and scarring may occur at injection and surgical sites. Implants may move and/or break through skin. Nonetheless, many are still willing to hear these risks.

In addition to these known risks, lip augmentation is considered extremely expensive by many. Costs for an injection can easily range from five hundred to one thousand dollars (US$). Because lip fullness will decrease over time, additional sessions—totaling multiple thousands of dollars—may be required to maintain fuller lips.

Because of the multitude of risks associated with lip augmentation, some have turned to using a nonsurgical device, commonly known as a lip pump, to increase lip size. These types of lip enlargement devices use some form of suction, i.e. negative pressure, applied over both lips to increase blood flow and cause lip size to temporarily increase. Although useful for their intended purpose, currently available lip pumps can have negative side effects. After use, some lip pumps will leave unattractive marks, bruising, indentations, and grooves around a user's lip area. In addition, some lip pumps continuously apply suction to both lips. Unfortunately, these types of lip pumps can cause misshaped lips and fail to address various lip problems. A user who only wants to increase a smaller lower lip, for example, may come away with a “ducky” upper lip that appears similar to a duck bill.

Considering the limitations of known lip pumps and the numerous risks associated with lip augmentation, the need for improved lip enhancement and enlargement devices is clear. There are multiple inventions that have been proposed in prior art in similar domain. For instance, a Lip enhancement and enlargement device bearing U.S. Pat. No. 1,068,2278B2 is issued to Thienna Ho. The patent is on a lip enhancement and enlargement device includes a suction element and a lip shaper coupled to the suction element. A lip shaper may include one or more features that allow the device to shape and contour lips. A lip shaper may include one or more contouring elements that change the shape of an upper lip, for example. A lip shaper may also include shaping and contouring elements that form a fuller, unitary lip lobe or two fuller, lip lobes. Lip enhancement and enlargement kits and methods are also disclosed.

Another patent on Lip augmentation device bearing US patent 2,007,0016277A1 is issued to Power Paper Ltd. The present invention is of a method of facilitating lip augmentation, the method comprising treating the lip area with an electricity generating device. The present invention further provides devices and kits for lip augmentation.

Another patent on Lip suction device and related methods hearing U.S. Pat. No. 8,858,472B2 is issued to Et Al Beauty LLC. The patent is on a lip suction device. A rigid hollow form has an opening at a first end of the rigid hollow form and a cavity therein extending from the first end to a second end of the rigid hollow form. The rigid hollow form has an outer surface and a thickness at the opening between the outer surface and a surface of the cavity. The opening has an edge with a rounded shape, across the thickness at the opening, which is configured to form a substantially air-tight seal between a user's face and the edge when the rigid hollow form is pressed against the user's face near the user's lips. The rigid hollow form is configured so that when the user draws air into the user's mouth after forming a substantially air-tight seal, the user's lips are drawn into the rigid hollow form towards the second end of the rigid hollow form.

Anomer patent on Transoral methods and compositions for wrinkle reduction and cosmetic lip and facial augmentation hearing U.S. Pat. No. 9,238,153B2 is issued to Sunny BioDiscovery Inc. The purpose of this patent is to teach the methods of application and compositions of cosmetic and therapeutic products for reducing wrinkle appearance and augmenting certain compartments of the facial integument of a mammal, especially a human. These methods and compositions utilize the hypodermis targeted release from bioadhesive oral films, pastes or patches applied to oral mucosa or teeth for the purpose of improving the appearance or health of the lips and facial skin.

A US patent on Lip balm bearing U.S. Pat. No. 9,517,196B2 is issued to ROBELL RESEARCH Inc Robell Research. The lip balm product having a synthetic ceramide, an anti-irritant compound, sodium hyaluronate, cosmetic wax, boswellic acids and/or esters and/or salts thereof, extract of the Portuluca pilosa plant, matrikin-mimetic peptide, dehydrated microspheres of marine collagen and glycosaminoglycans, and mica along with optional lip balm formulation acceptable carriers and/or solvents. Where desired, the lip balm may further contain medicinal agents suitable for the treatment of various conditions of the lip.

There are multiple solutions that have been presented in prior art. However, these solutions are limited and restricted to their conventional systems. The current invention is focused on presenting an assembly which is directed to a small device Lip Plumper Lip Gloss, which will allow individuals to have a safe, sanitary, effective tool to help shape, contour and smooth the lips skin following the procedures. Furthermore, it will allow individuals to administer this ‘self-massage’ to enhance their overall look and feel of their lip plumper that will provide the best outcome of without any procedure and obtain the desired result. The present invention fulfills this need and provides further related advantages, as described in the following summary.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of Lip Plumper and Lip Gloss tool which can be used for lip enhancement and enlargement.

It is also the primary objective of the invention to provide an assembly which helps to Lip augmentation device for enhancing the lips and can include plumping, reshaping, such as enlargement or lifting of the lips.

It is another objective of the invention to promote the enhancement of an invasive technique and although not complex, which doesn't includes the risks of any surgical procedure.

It is also the object of the invention to provide an assembly which helps to provide lip oil allowing to help exquisite and luster lips by diluting unattractive wrinkles.

It is also the object of the invention to provide an assembly which can be easily used on lips which appear more voluptuous right after applying it, perfect for an instant dramatic pout.

It is further the objective of the invention to provide an assembly which keeps lips hydrated and simultaneously plump them up, by pulling moisture from the environment or trapping water that would otherwise evaporate from the skin, giving the hydrated look with a non-sticky formula.

It is also the objective of the invention to provide a smart assembly which provide ease of use and convenience to the user.

Thus, it is the objective to provide a new and improved assembly which is a combination of Lip Plumper Lip Gloss. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to a safe and easy to apply assembly to achieve lip enhancement and fullness without requirement of any physical procedure.

The assembly as per its illustrative embodiment is made of high-quality Hydrogenated Polyisobutene, Mineral Oils, Isopropyl Myristate, Hydrogenated Styrene/Isoprene Copolymer, Ginger Perfume and Vanillyl Butyl Ether. This combination id developed to achieve exquisite and luster lips by diluting unattractive wrinkles.

The Hydrogenated Polyisobutene is a synthetic oil that is used as a mineral oil substitute. It is an emollient & moisturizer and prevents water loss. The Polyisobutene dries to form a thin coating on the skin, hair or nails. Hydrogenated Polyisobutene acts as a lubricant on the skin's surface, which gives the skin a soft and smooth appearance. The ingredient of Hydrogenated Polyisobutene keeps the lips hydrated and simultaneously plump them up. The product also focuses on increasing lip volume by pulling moisture from the environment or trapping water that would otherwise evaporate from the skin, giving the ultimate look sexy hydrated look with a non-sticky formula to lips.

Moreover, the Isopropyl Myristate is a synthetic oil used as an emollient, thickening agent, or lubricant in beauty products. Composed of Isopropyl Alcohol (a propane derivative) and Myristic Acid (a naturally occurring fatty acid). Isopropyl Myristate is a popular cosmetic and pharmaceutical ingredient. It has an incredible ability to moisturize the skin, strengthening the skin's natural moisture barrier, and helping it stay hydrated to reduce moisture loss. As such, Isopropyl Myristate is a natural emollient choice for many creams, moisturizers, and serums on the market. The inclusion of Isopropyl Myristate allows the moisture to lock in and remain fresh and hydrated. By applying the proposed formula, the lips absorb the moisture and plump up, giving the ultimate sunkissed, chilled and refresh look to lips.

Hydrogenated Styrene/Isoprene Copolymer functions in skincare and cosmetics as a viscosity controlling ingredient. This Modified silicones such as polyether/alkyl co-modified silicones are added to improve texture and comfort. The mixture of the solid resin with the solid polymeric material result in a softer material with good adhesion and less tack on the skin.

Other minerals and perfume agents are added in proportionate quantities to provide complete nourishment and enhanced user experience.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

I: A method of facilitating lip augmentation, the method comprising treating the lip area with a chemical formula comprising:

| | |
|---|---|
| Hydrogenated Polyisobutene | 32.4% |
| Mineral Oil | 32.2% |
| Isopropyl Myristate | 30.1% |
| Hydrogenated Styrene/Isoprene Copolymer | 5% |
| Ginger Perfume | 0.1% |
| Vanillyl Butyl Ether | 0.1% |

II: The method of claim I, wherein the improvement in aesthetic appearance is selected from the group consisting of:
a. improvement in lip color;
b. improvement in lip fullness;
c. improvement in lip dryness;
d. improvement in lip clarity;
e. reduction in the number vertical lip lines;
f. reduction in the depth of vertical lip lines;
g. improvement in lip dryness appearance; and
h. combinations thereof.
